(19) United States
(12) Patent Application Publication
(10) Pub. No.: US 2025/0339372 A1
(43) Pub. Date: Nov. 6, 2025

(54) PHARMACEUTICAL COMPOSITION COMPRISING A GLP-1-AGONIST AND METHIONINE

(71) Applicant: Sanofi-Aventis Deutschland GmbH, Frankfurt (DE)

(72) Inventors: Anette Brunner-Schwarz, Frankfurt (DE); Werner Mueller, Frankfurt (DE); Verena Siefke-Henzler, Frankfurt (DE)

(21) Appl. No.: 19/187,149

(22) Filed: Apr. 23, 2025

Related U.S. Application Data
(63) Continuation of application No. 17/747,723, filed on May 18, 2022, now Pat. No. 12,303,598.
Application No. 17/747,723 is a continuation of application No. 16/921,422, filed on Jul. 6, 2020, now abandoned.
Application No. 16/921,422 is a continuation of application No. 16/013,617, filed on Jun. 20, 2018, now abandoned.
Application No. 16/013,617 is a continuation of application No. 15/595,929, filed on May 15, 2017, now Pat. No. 10,028,910.
Application No. 15/595,929 is a continuation of application No. 13/509,507, filed on Jul. 30, 2012, now Pat. No. 9,707,176.

(30) Foreign Application Priority Data
Nov. 13, 2009 (DE) 102009052832.6
Mar. 18, 2010 (DE) 102010011919.9

(51) Int. Cl. A61K 9/08; A61K 9/00; A61K 38/22; A61K 38/26; A61K 47/10; A61K 47/20; A61P 3/10

(52) U.S. Cl. CPC A61K 9/08; A61K 9/0019; A61K 38/2278; A61K 38/26; A61K 47/10; A61K 47/20; A61P 3/10

(57) ABSTRACT

A liquid composition comprising a GLP-1 agonist or/and a pharmacologically tolerable salt thereof and, optionally, at least one pharmaceutically acceptable excipient, wherein the composition comprises methionine, as add-on therapy with metformin and/or with long-acting insulin/insulin derivates where appropriate.

### Pharmaceutical Composition Comprising a GLP-1 Agonist and Methionine

The present application relates to a liquid composition comprising a GLP-1 agonist or/and a pharmacologically tolerable salt thereof and, optionally, at least one pharmaceutically acceptable excipient, wherein the composition comprises methionine.

The present application further relates to the composition according to the present invention for treating diabetes mellitus. The present application further relates to the use of a composition according to the present invention in the manufacture of a pharmaceutical for treating diabetes mellitus. The present application further relates to a method for manufacturing a composition according to the present invention, comprising formulating a GLP-1 agonist or/and a pharmacologically tolerable salt thereof with methionine and, optionally, at least one pharmaceutically acceptable excipient. The present application further relates to a method for treating a patient with a composition according to the present invention, comprising administering the composition to the patient.

Customary compositions of GLP-1 compounds comprise a tonicity modifier, a buffer for adjusting the pH, and a preservative.

WO2001/04156 (Zealand Pharmaceuticals) discloses a liquid composition of Ser³⁹-exendin-4(1-39)-NH₂), sodium dihydrogenphosphate, and preservatives.

WO 2004/035623 (Zealand Pharmaceuticals) discloses a liquid composition comprising a stabilized exendin, 50 mM histidine, 100 to 200 mM sucrose, mannitol or other acceptable sugar, 20 mM methionine, 20 mM asparagine-glutamine or Asp, at a pH of 5.3. Stabilization is effected by certain modifications of the amino acid building blocks of exendin-4(1-39), for example, at positions Gln13, Met14, Trp25, or Asn28.

WO 2005/021022 (Novo Nordisk) discloses a liquid composition comprising acetylated GLP-1, phenol as a preservative, mannitol and glycerol as a tonicity modifier, and, optionally, a buffer.

WO 2006/051110 (Novo Nordisk) discloses liquid compositions comprising liraglutide (GLP-1 compound), poloxamer 188 or poloxamer 407 (Pluronic F-127) as a surface-active substance, phenol, propylene glycol, and sodium phosphate (pH 7.7). Addition of poloxamer-188 or poloxamer-407 led to stabilization.

Exendins are a group of peptides which can lower blood glucose concentrations. Exendins have a certain similarity to the sequence of GLP-1 (7-36) (53%, Goke et al. J. Biol Chem 268, 19650-55). Exendin-3 and exendin-4 stimulate an increase in cellular cAMP production in the acinar cells of the guinea pig pancreas by interacting with exendin receptors (Raufman, 1996, Reg. Peptides 61:1-18). Exendin-3, in contrast to exendin-4, effects an increase in the release of amylase in the acinar cells of the pancreas. Exendins act as GLP-1 agonists.

Glucagon-like peptide 1 (GLP-1) is an endocrine hormone which enhances the insulin response following oral intake of glucose or fat. In general, GLP-1 lowers glucagon concentrations, slows gastric emptying, stimulates (pro)insulin synthesis, enhances sensitivity to insulin, and stimulates insulin-independent glycogen synthesis (Holst (1999), Curr. Med. Chem 6:1005, Nauck et al. (1997) Exp Clin Endocrinol Diabetes 105:187, Lopez-Delgado et al. (1998) Endocrinology 139:2811). Human GLP-1 has 37 amino acid residues (Heinrich et al., Endocrinol. 115:2176 (1984), Uttenthal et al., J Clin Endocrinol Metabol (1985) 61:472). Active fragments of GLP-1 include GLP-1 (7-36) and GLP-1 (7-37).

Exendin-3, exendin-4 and exendin agonists have been proposed for treating diabetes mellitus and preventing hyperglycemia, by reducing gastric motility and gastric emptying (U.S. Pat. No. 5,424,286 and WO98/05351).

Exendin analogs can be characterized by amino acid substitutions and/or C-terminal truncation of the native exendin-4 sequence. Such exendin analogs are described in WO 99/07404, WO 99/25727, and WO 99/25728.

Solid-phase synthesis of AVE0010 is described in WO 01/04156 A1. AVE0010 has the sequence: desPro³⁶exendin-4(1-39)-Lys₆-NH₂. This substance is published as SEQ ID NO:93 in WO 01/04156:

(SEQ ID NO: 1)
H-G-E-G-T-F-T-S-D-L-S-K-Q-M-E-E-E-A-V-R-L-F-I-E-W-
L-K-N-G-G-P-S-S-G-A-P-P-S-K-K-K-K-K-K-NH₂

Exendin-4 (39 AS) has the sequence:

(SEQ ID NO: 2)
H-G-E-G-T-F-T-S-D-L-S-K-Q-M-E-E-E-A-V-R-L-F-I-E-W-
L-K-N-G-G-P-S-S-G-A-P-P-P-S-NH₂

Exendin-3 has the sequence (J. Bio. Chem., 267, 1992, 7402-7405):

(SEQ ID NO: 3)
H-His-Ser-Asp-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-NH₂

GLP-1 has the sequence:

(SEQ ID NO: 4)
H-A-E-G-T-F-T-S-D-V-S-S-Y-L-E-G-Q-A-A-K-E-F-I-A-W-
L-V-K-G-R-NH₂

It is an object of the present invention to increase the stability of liquid formulations comprising a GLP-1 agonist. More particularly, it is an object of the present invention to improve physical and chemical integrity. We have found that this object is achieved by formulating the GLP-1 agonist with methionine.

It was found that methionine is able to increase the storage stability of a composition comprising a GLP-1 agonist such as AVE0010. Methionine does not affect the physical integrity of these compositions.

It was found that, surprisingly, the addition of methionine is able to improve the storage stability of a composition according to the present invention by reducing the proportion of oxidation products of methionine, of proteins of high molecular weight, and of total impurities. These parameters are, individually or together, a measure of the chemical integrity of the compositions.

It was further found that, surprisingly, the biological activity of the compositions according to the present invention is increased by the addition of methionine.

The stability of pharmaceutically active polypeptides can be impaired by various mechanisms. These include pH, temperature, light, and the effects of certain constituents.

A range of customary constituents of formulations of GLP-1 agonists can be disadvantageous for the chemical or/and physical integrity and the storage stability of formulations which comprise a GLP-1 agonist. These are, for example, polysorbate 20, polysorbate 80, poloxamer 188, benzalkonium chloride, and lysine. The compositions according to the present invention are therefore preferably free of these constituents.

The present invention accordingly provides for a liquid composition comprising a GLP-1 agonist or/and a pharmacologically tolerable salt thereof and, optionally, at least one pharmaceutically acceptable excipient, wherein the composition comprises methionine.

The composition according to the present invention preferably comprises methionine in an amount ranging from 0.5 mg/mL to 20 mg/mL, more preferably in an amount ranging from 1 mg/mL to 5 mg/mL. Methionine in the D-form can be used. Likewise, methionine in the L-form can be used. Likewise, mixtures of the D-form and the L-form in any desired proportions can be used.

More particularly, the composition according to the present invention is free of surfactants, such as polyols and partial and fatty acid esters and ethers of polyhydric alcohols such as those of glycerol and sorbitol. The compositions according to the present invention are more particularly free of partial and fatty acid esters and ethers of glycerol and sorbitol selected from the group consisting of Span®, Tween®, Myrj®, Brij®, Cremophor®. Furthermore, the compositions according to the present invention are more particularly free of polyols selected from the group consisting of polypropylene glycols, polyethylene glycols, poloxamers, Pluronics, Tetronics. More 10 particularly, the composition according to the present invention is free of at least one substance selected from the group consisting of polysorbate, polysorbate and poloxamer.

More particularly, the composition according to the present invention is substantially free, preferably free, of polysorbate, such as, for example, polysorbate 20.

More particularly, the composition according to the present invention is substantially free, preferably free, of polysorbate 80.

More particularly, the composition according to the present invention is substantially free, preferably free, of poloxamer, such as, for example, poloxamer 188.

More particularly, the composition according to the present invention is substantially free, preferably free, of benzalkonium chloride.

More particularly, the composition according to the present invention is substantially free, preferably free, of histidine.

More particularly, the composition according to the present invention is substantially free, preferably free, of EDTA, more particularly sodium EDTA.

The composition according to the present invention can comprise one or more substances which are customarily used to buffer the pH (buffer substances).

Examples of such buffer substances are acetate, citrate, and phosphate, for example, in amounts of up to 5 mg/ml, up to 4 mg/ml, up to 3 mg/ml, or up to 2 mg/ml.

The composition according to the present invention can, likewise, be substantially free of buffer substances. Likewise, the composition according to the present invention can be free of buffer substances.

The composition according to the present invention can be substantially free of citrate, acetate, and/or phosphate, or else free of citrate, acetate, and/or phosphate.

More particularly, the composition according to the present invention is substantially free, preferably free, of histidine and sodium EDTA.

More particularly, no insulin is present in the composition according to the present invention.

The pharmaceutical composition of the present invention can have an acidic or physiological pH. An acidic pH range is preferably in the range of pH 1-6.8, pH 3.5-6.8, or pH 3.5-5. A physiological pH is preferably in the range of pH 2.5-8.5, more preferably pH 4.0 to 8.5, even more preferably pH 4.0 to 6.0. Especially preferred is a pH of approximately 4.5. For pH adjustment, physiologically safe dilute acids (typically HCl) and alkalis (typically NaOH) are suitable.

The composition according to the present invention can comprise a suitable preservative. Suitable preservatives are, for example, phenol, m-cresol, benzyl alcohol, and/or p-hydroxybenzoate esters. m-Cresol is preferred.

Furthermore, the composition according to the present invention can comprise suitable tonicity modifiers. Suitable tonicity modifiers are, for example, glycerol, dextrose, lactose, sorbitol, mannitol, glucose, NaCl, calcium or magnesium compounds such as CaCl₂, etc. The concentrations of glycerol, dextrose, lactose, sorbitol, mannitol, and glucose are customarily in the range of 100-250 mM, NaCl in a concentration of up to 150 mM. Glycerol is preferred.

More particularly, the composition is intended for parenteral administration. The composition according to the present invention is preferably an injectable composition, more preferably for subcutaneous injection. More particularly, the composition of the present invention is suitable for injection once a day.

More particularly, the formulation according to the present invention has, after storage for 1 month, 2 months, 4 months, or 6 months at a temperature of +5° C. or 25° C., an activity of at least 80%, at least 90%, at least 95%, or at least 98% of the activity at the start of storage.

In the present application, “activity” means the activity of the GLP-1 agonist which is used in the formulation according to the present invention. Methods for determining the activity of a GLP-1 agonist are known to a person skilled in the art.

Preferably, the composition according to the present invention has a biological activity of GLP-1 agonist of at least 89% or at least 90% after storage for 6 months at 25° C. The composition according to the present invention preferably has a biological activity of GLP-1 agonist of at least 45% or at least 50% after storage for 6 months at 40° C.

More particularly, the formulation according to the present invention exhibits chemical integrity after storage for 1 month, 2 months, 3 months, 4 months, or 6 months. Chemical integrity means, more particularly, that after storage at a temperature of +5° C., 25° C., or 40° C. the formulation comprises at least 80%, at least 90%, at least 95%, or at least 98% of the active substance, compared with the start of storage, in a substantially chemically unchanged form.

Chemical integrity can mean the chemical integrity of the GLP-1 agonist. GLP-1 agonists may comprise a methionine residue (e.g. position 14 in AVE0010). Chemical integrity of the GLP-1 agonist means, more particularly, that oxidation of the methionine residue is prevented. Here, chemical integrity means, more particularly, that the proportion of oxidized methionine with respect to the entire methionine content of the GLP-1 agonist after storage for 1, 2, 3, 4, or 6 months is below 0.7%, below 0.6%, below 0.5%, below 0.4%, or below 0.3%. Storage can be effected, for example, at 5° C., 25° C., or 40° C. Storage for 6 months at 5° C. is preferred, in which case the proportion of oxidized methionine is below 0.3%. Likewise, storage for 6 months at 25° C. is preferred, in which case the proportion of oxidized methionine is below 0.7%, below 0.6%, below 0.5%, below 0.4%, or below 0.3%. Likewise, storage for 6 months at 40° C. is preferred, in which case the proportion of oxidized methionine is below 1%, below 0.7%, below 0.6%, below 0.5%, below 0.4%, or below 0.3%.

Chemical integrity can mean a very low proportion of total impurities in the formulation according to the present invention. The proportion of total impurities with respect to the entire mass of the GLP-1 agonist present in the formulation after storage for 6 months at 40° C. is more particularly below 50%, below 10% after storage at 25° C., or/and below 1.8% after storage at 5° C.

Chemical integrity can mean a very low proportion of proteins of high molecular weight in the formulation according to the present invention. The proportion of proteins of high molecular weight with respect to the entire mass of the GLP-1 agonist present in the formulation after storage for 6 months at 40° C. is more particularly below 5%, below 4%, below 3%, or below 2%. After storage for 6 months at 25° C., the proportion of proteins of high molecular weight with respect to the entire mass of the GLP-1 agonist present in the formulation is more particularly below 0.8%, below 0.7%, or below 0.6%.

More particularly, the formulation according to the present invention exhibits physical integrity after storage for 1 month, 2 months, 4 months, or 6 months. Physical integrity means, more particularly, that after storage at a temperature of +5° C., 25° C., or 40° C. the formulation comprises at least 80%, at least 90%, at least 95%, or at least 98% of the active substance, compared with the start of storage, in a substantially physically unchanged form.

Physical integrity can mean the integrity of the GLP-1 agonist. Physical integrity means, more particularly, that the GLP-1 agonist does not form aggregates, such as, for example, fibrils.

The GLP-1 agonist is preferably selected from the group consisting of exendin-3 and analogs and derivates thereof, exendin-4 and analogs and derivates thereof, and in which case the GLP-1 agonist is more preferably selected from the group consisting of AVE0010 and exendin-4.

Exendin-3, analogs and derivates of exendin-3, exendin-4, and analogs and derivates of exendin-4 can be found in WO 01/04156, WO 98/30231, U.S. Pat. No. 5,424,286, EP application Ser. No. 99/610,043.4, and WO 2004/005342. These documents are incorporated herein by reference. The exendin-3, exendin-4, and analogs and derivates thereof described in these documents can be synthesized by means of the methods described therein, after which modifications are optionally carried out.

The sequences of AVE0010 (SEQ ID NO:1), exendin-4 (SEQ ID NO:2), and exendin-3 (SEQ ID NO:3) show a high degree of similarity. The sequences of AVE0010 and exendin-4 are identical at positions 1-37. Sequence 1-39 from exendin-4 is at 37 of the 39 positions (94%) identical to the exendin-3 sequence at positions 48-86. With reference to the sequences, a person skilled in the art can readily convert the positions specified herein, which relate to a particular sequence (e.g. to the sequence of AVE0010 or exendin-4), to other sequences.

Analogs and derivates of exendin-3 or/and exendin-4 contain more particularly a modified amino acid sequence. For example, single amino acids can be deleted (e.g. desPro36, desPro37, desAsp28, desMet(O)14 in exendin-4 and the corresponding positions in exendin-3). Likewise, single positions can be substituted (e.g. Met(O)¹⁴, Trp(O₂)²⁵, IsoAsp²⁸, Asp²⁸Pro³⁸ in exendin-4 and the corresponding positions in exendin-3), in which case unnatural amino acids such as Met(O) (methionine sulfoxide or methionine sulfone), Trp(O₂) (N-formylkynurenine), or/and IsoAsp (β-aspartate or isoaspartate) can also be used. Unnatural amino acids can be readily inserted, in the form of corresponding amino acid building blocks, into the sequence. Furthermore, the C-terminus or/and the N-terminus can be modified, for example, by an additional sequence such as -(Lys)-, -(Lys)₂-, -(Lys)₃-, -(Lys)₄-, -(Lys)₅-,-(Lys)₆-, -Asn-(Glu)₅-, in which case -(Lys)₄-, -(Lys)₅-, -(Lys)₆-, -Asn-(Glu)₅- are preferred. The carboxyl group at the C-terminus is preferably modified to an amide group (—NH₂). Optionally, modification of the C-terminus or/and of the N-terminus is carried out as a further step after completion of synthesis.

Pharmaceutically tolerable salts can be manufactured in a further step after completion of the synthesis cycles of the method according to the present invention. The manufacture of pharmaceutically tolerable salts of peptides is known to a person skilled in the art. A preferred pharmaceutically tolerable salt is acetate.

The GLP-1 agonist is preferably selected from the group consisting of exendin-4, analogs and derivates of exendin-4, and pharmacologically tolerable salts thereof.

A further preferred GLP-1 agonist is an analog of exendin-4 selected from the group consisting of:
H-desPro36-exendin-4-Lys₆-NH₂,
H-des(Pro36,37)-exendin-4-Lys₄-NH₂,
H-des(Pro36,37)-exendin-4-Lys₅-NH₂ and pharmacologically tolerable salts thereof.

A further preferred GLP-1 agonist is an analog of exendin-4 selected from the group consisting of:
desPro³⁶[Asp²⁸]exendin-4 (1-39),
desPro³⁶[IsoAsp²⁸]exendin-4 (1-39),
desPro³⁶[Met(O)¹⁴,Asp²⁸]exendin-4 (1-39),
desPro³⁶[Met(O)¹⁴,IsoAsp²⁸]exendin-4 (1-39),
desPro³⁶[Trp(O₂)²⁵ ,Asp²⁸]exendin-2 (1-39),
desPro³⁶[Trp(O₂)²⁵,IsoAsp²⁸]exendin-2 (1-39),
desPro³⁶[Met(O)¹⁴Trp(O₂)²⁵,Asp²⁸]exendin-4 (1-39),
desPro³⁶[Met(O)¹⁴Trp(O₂)²⁵,IsoAsp²⁸]exendin-4 (1-39) and pharmacologically tolerable salts thereof.

A further preferred GLP-1 agonist is an analog of exendin-4 selected from a group as described in the previous paragraph, wherein the peptide-Lys₆-NH₂ is attached to the C-termini of the analogs of exendin-4.

A further preferred GLP-1 agonist is an analog of exendin-4 selected from the group consisting of:
H-(Lys)₆-desPro³⁶[Asp²⁸]exendin-4(1-39)-Lys₆-NH₂,
desAsp²⁸Pro³⁶, Pro³⁷, Pro³⁸exendin-4(1-39)-NH₂,
H-(Lys)₆-desPro³⁶,Pro³⁷,Pro³⁸[Asp²⁸]exendin-4(1-39)-NH₂,
H-Asn-(Glu)₅desPro³⁶,Pro³⁷,Pro³⁸[Asp²⁸]exendin-4(1-39)-NH₂,
desPro³⁶,Pro³⁷,Pro³⁸[Asp²⁸]exendin-4 (1-39)-(Lys)₆-NH₂,
H-(Lys)₆-desPro³⁶,Pro³⁷,Pro³⁸[Asp²⁸]exendin-4(1-39)-(Lys)₆-NH₂,
H-Asn-(Glu)₅-desPro³⁶,Pro³⁷,Pro³⁸[Asp²⁸]exendin-4(1-39)-(Lys)₆-NH₂,
H-(Lys)₆-desPro³⁶[Trp(O₂)²⁵,Asp²⁸]exendin-4(1-39)-Lys₆-NH₂,
H-desAsp²⁸ Pro³⁶,Pro³⁷,Pro³⁸[Trp(O₂)²⁵]exendin-4(1-39)-NH₂,
H-(Lys)₆-desPro³⁶,Pro³⁷,Pro³⁸[Trp(O₂)²⁵,Asp²⁸]exendin-4(1-39)-NH₂,
H-Asn-(Glu)₅-desPro³⁶,Pro³⁷,Pro³⁸[Trp(O₂)²⁵, Asp²⁸]exendin-4(1-39)-NH₂,
desPro³⁶,Pro³⁷,Pro³⁸[Trp(O₂)²⁵, Asp²⁸]exendin-4(1-39)-(Lys)₆-NH₂,
H-(Lys)₆-desPro³⁶,Pro³⁷, Pro³⁸[Trp(O₂)²⁵, Asp²⁸]exendin-4(1-39)-(Lys)₆-NH₂,
H-Asn-(Glu)₅-desPro³⁶,Pro³⁷,Pro³⁸[Trp(O₂)²⁵, Asp²⁸]exendin-4(1-39)-(Lys)₆-NH₂,
H-(Lys)₆-desPro³⁶[Met(O)¹⁴,Asp²⁸]exendin-4(1-39)-(Lys)₆-NH₂,
desMet(O)¹⁴Asp²⁸Pro³⁶,Pro³⁷,Pro³⁸exendin-4 (1-39)-NH₂,
H-(Lys)₆-desPro³⁶,Pro³⁷,Pro³⁸[Met(O)¹⁴,Asp²⁸]exendin-4(1-39)-NH₂,
H-Asn-(Glu)₅-desPro³⁶,Pro³⁷,Pro³⁸[Met(O)¹⁴,Asp²⁸]exendin-4(1-39)-NH₂,
desPro³⁶,Pro³⁷,Pro³⁸[Met(O)¹⁴,Asp²⁸]exendin-4(1-39)-(Lys)₆-NH₂,
H-(Lys)₆-desPro³⁶,Pro³⁷,Pro³⁸[Met(O)¹⁴,Asp²⁸]exendin-4(1-39)-Lys₆-NH₂,
H-Asn-(Glu),-desPro³⁶,Pro³⁷,Pro³⁸[Met(O)¹⁴,Asp²⁸]exendin-4(1-39)-(Lys)₆-NH₂,
H-(Lys)₆-desPro³⁶[Met(O)¹⁴, Trp(O₂)²⁵,Asp²⁸]exendin-4(1-39)-Lys₆-NH₂,
desAsp²⁸Pro³⁶,Pro³⁷,Pro³⁸[Met(O)¹⁴, Trp(O₂)²⁵]exendin-4(1-39)-NH₂,
H-(Lys)₆-desPro³⁶,Pro³⁷,Pro³⁸[Met(O)¹⁴, Trp(O₂)²⁵,Asp²⁸]exendin-4(1-39)-NH₂,

H-Asn-(Glu)₅-desPro³⁶,Pro³⁷,Pro³⁸[Met(O)¹⁴, Asp²⁸]exendin-4(1-39)-NH₂,
desPro³⁶,Pro³⁷,Pro³⁸[Met(O)¹⁴, Trp(O₂)²⁵,Asp²⁸]exendin-4(1-39)-(Lys)₆-NH₂,
H-(Lys)₆-desPro³⁶,^{Pro37},Pro³⁸[Met(O)¹⁴,Trp(O₂)²⁵,Asp²⁸]exendin-4(1-39)-(Lys)₆-NH₂,
H-Asn-(Glu)₅-desPro³⁶, Pro³⁷, Pro³⁸[Met(O)¹⁴,Trp(O₂)²⁵,Asp²⁸]exendin-4(1-39)-(Lys)₆-NH₂ and pharmacologically tolerable salts thereof.

Likewise, the GLP-1 agonist can be selected from the group consisting of GLP-1 and analogs and derivates of GLP-1. A further preferred GLP-1 agonist is selected from the group consisting of Arg³⁴, Lys²⁶(N^{ε}(Υ-glutamyl(N^{α}-hexadecanoyl)))GLP-1(7-37) [liraglutide] and a pharmacologically tolerable salt thereof.

A further preferred GLP-1 agonist is AVE0010. AVE0010 has the sequence desPro³⁶exendin-4(1-39)-Lys₆-NH₂ (SEQ ID NO:1). Likewise, pharmacologically tolerable salts of AVE0010 are preferred.

The GLP-1 agonist, for example AVE0010, is more particularly used in an amount ranging from 0.01 mg/ml to 0.5 mg/ml or 0.05 mg/ml to 1.5 mg/ml.

In a particular embodiment, the formulation according to the present invention comprises the following constituents:
(a) desPro³⁶exendin-4(1-39)-Lys₆-NH₂ (e.g. approximately 0.1 mg/mL),
(b) sodium acetate trihydrate (approximately 3.5 mg/ml),
(c) m-cresol (approximately 2.7 mg/mL),
(d) L-methionine (approximately 3 mg/mL),
(e) 85% glycerol (approximately 18 mg/mL),
(f) approximately 0.1 N hydrochloric acid, if adjustment to a pH of approximately 4.5 is required,
(g) approximately 0.1 N NaOH solution, if adjustment to a pH of approximately 4.5 is required, and
(h) water.

More particularly, the formulation according to the present invention consists of the constituents mentioned in (a) to (h).

In the present application, “approximately” means that the constituents can be present, for example, within the ranges of ±10, ±20, or ±30% around the specified values in the compositions according to the present invention.

If the composition according to the present invention comprises more than one GLP-1 agonist, then these GLP-1 agonists are selected independently of one another.

Suitable packaging for the composition according to the present invention is, for example, a syringe or a glass vessel with a suitable closure, from which individual therapeutically effective doses can be withdrawn as needed. Equally suitable are injection pens for administering doses; such pens comprise a container (e.g. a cartridge) which contains a pharmaceutical composition according to the present invention.

The present invention further provides for a method for treating a patient with a composition according to the present invention, comprising administering the composition to the patient.

The composition according to the present invention is intended more particularly for treating diabetes mellitus, more particularly for treating type I or type II diabetes mellitus. Further possible indications are symptoms which are associated with diabetes mellitus. Preferably, the composition according to the present invention is used to control the fasting, postprandial, or/and postabsorptive plasma glucose concentration, to improve glucose tolerance, to prevent hypoglycemia, to prevent functional loss of the β-cells of the pancreas, to effect weight loss, or/and to prevent weight gain.

The present invention further provides for the use of a composition according to the present invention in the manufacture of a pharmaceutical for treating diabetes mellitus, more particularly type I or type II, or/and the symptoms associated with it, as described herein.

The present invention further provides a method for manufacturing a composition according to the present invention, comprising formulating a GLP-1 agonist or/and a pharmacologically tolerable salt thereof with methionine and, optionally, at least one pharmaceutically acceptable excipient.

The present invention further provides for the use of the composition according to the present invention together with the administration of metformin, sulfonylurea, or glitazones, a long-acting insulin/insulin derivate, and/or a combination thereof, more particularly as an add-on therapy in the administration of metformin.

The present invention further provides for the use of the composition according to the present invention in patients whose blood sugar levels cannot be controlled sufficiently by the administration of metformin, sulfonylurea, or glitazones, a long-acting insulin/insulin derivate, and/or a combination thereof.

The present invention further provides for the use of the composition according to the present invention in patients with type II diabetes as a supplement to a diet in order to improve blood sugar control.

More particularly, the composition comprises desPro³⁶ exendin-4(1-39)-Lys₆-NH₂ (AVE0010), liraglutide and/or a pharmacologically tolerable salt together with methionine and/or a pharmacologically tolerable salt.

More particularly, Lantus®, N^{εB29}-tetradecanoyl des (B30) human insulin, or Insuman® is useful as a long-acting insulin derivative.

Especially preferred is the add-on therapy with metformin and/or a long-acting insulin/insulin derivate and/or a pharmacologically tolerable salt thereof for treating type II diabetes and/or obesity, more particularly in patients who are younger than 50 years and/or have a body mass index of at least 30.

In the present invention, the add-on therapy involves more particularly the treatment of type II diabetes with metformin and AVE0010. Metformin and AVE0010 can be administered in a time interval of 24 hours. Metformin and AVE0010 can each be administered in a once-a-day dosage. Metformin and AVE0010 can be administered by means of different routes of administration. Metformin can be administered orally, AVE0010 subcutaneously.

Patients treated with the add-on therapy according to the present invention can have an HbA1c value in the range of 7% to 10%. They are preferably in the age range of 18 to 50 years.

The use in the add-on therapy according to the present invention is more particularly applicable to patients in whom type II diabetes cannot be sufficiently controlled with metformin alone.

More particularly, metformin is administered as follows: at least 1.0 g/day, preferably at least 1.5 g/day for 3 months.

The invention is further elucidated by the following examples and figures.

### LEGENDS

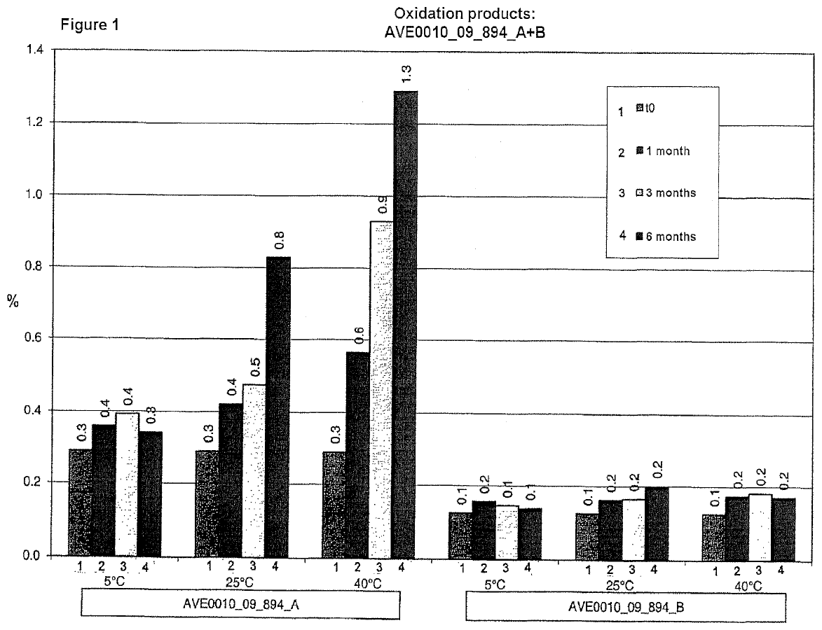

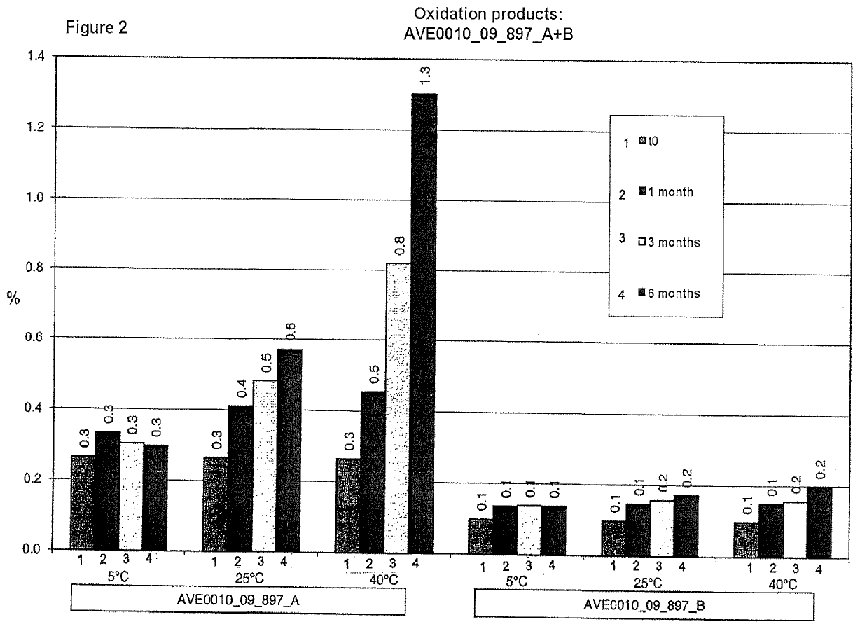

FIGS. 1 and 2 show the percentage content of oxidized methionine Met(ox) with respect to the entire methionine content of AVE0010 after storage at different temperatures. 1: start of storage t0. 2: storage for 1 month. 3: storage for 3 months. 3: storage for 6 months. FIG. 1: batch 894. FIG. 2: batch 897.

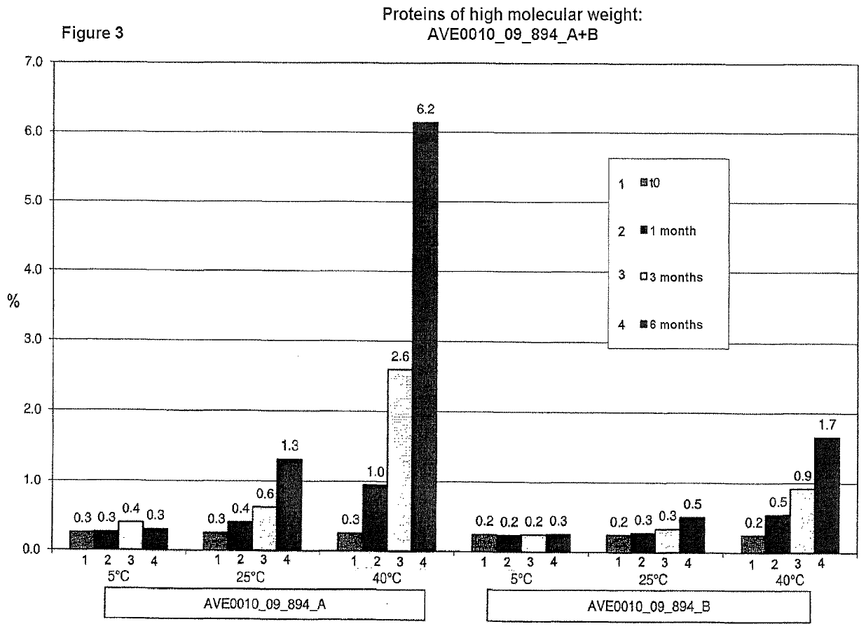

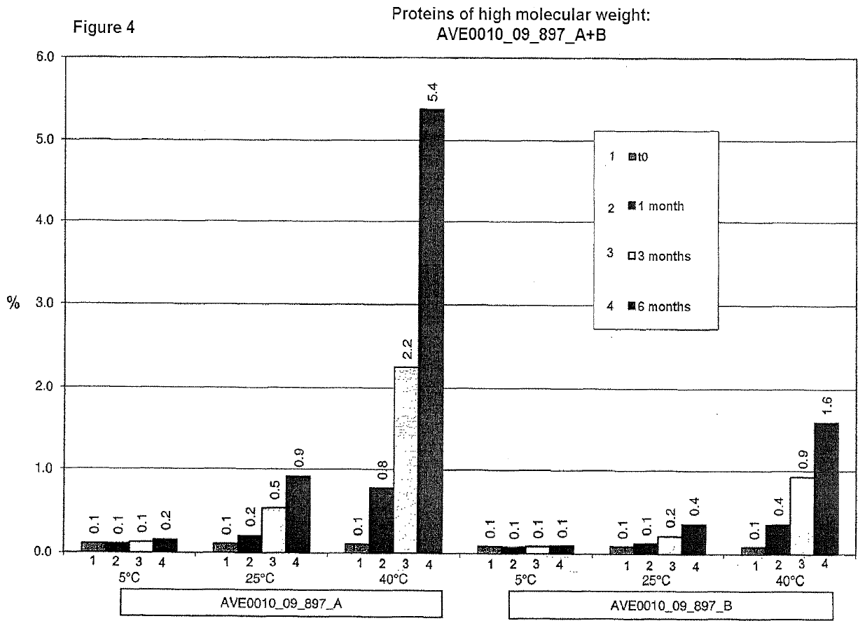

FIGS. 3 and 4 show the percentage content of protein impurities of high molecular weight (with respect to AVE0010) after storage at different temperatures. 1: start of storage t0. 2: storage for 1 month. 3: storage for 3 months. 3: storage for 6 months. FIG. 3: batch 894. FIG. 4: batch 897.

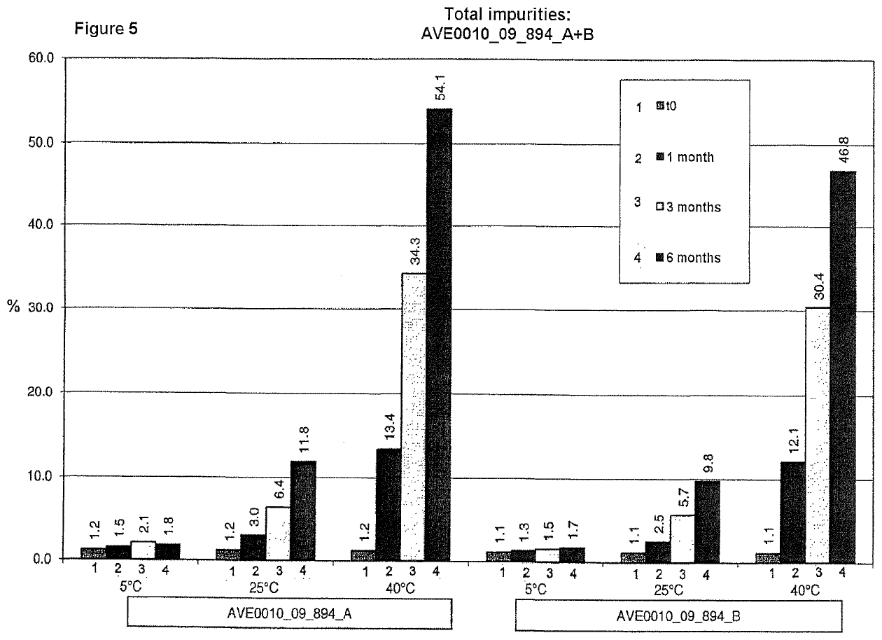

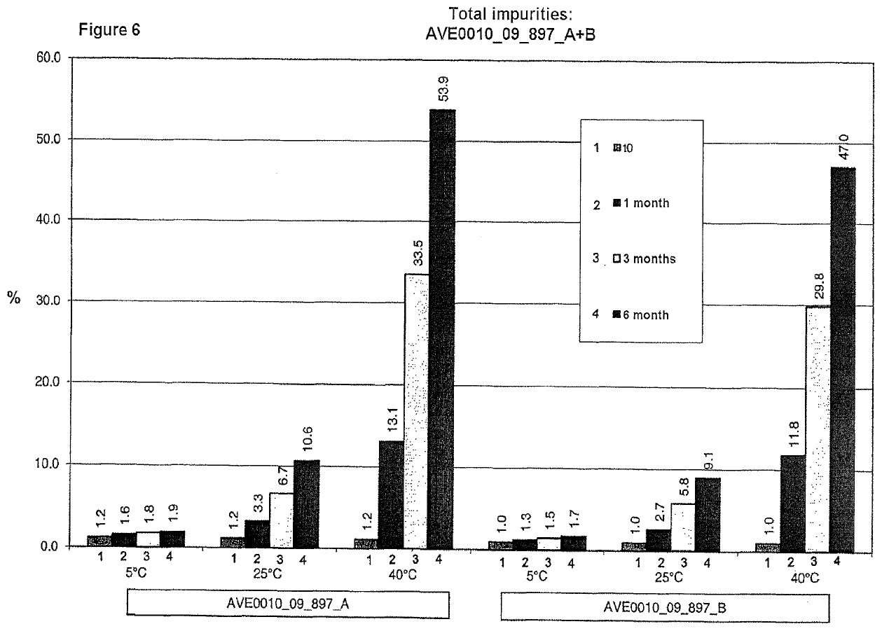

FIGS. 5 and 6 show the percentage content of all impurities (with respect to AVE0010) after storage at different temperatures. 1: start of storage t0. 2: storage for 1 month. 3: storage for 3 months. 3: storage for 6 months. FIG. 5: batch 894. FIG. 6: batch 897.

### EXAMPLE 1

### Liquid Composition Comprising AVE0010 and Methionine

The purpose of the study is the evaluation of the chemical or/and physical stability of formulations of AVE0010 (solution for injection, 0.1 mg/ml) with and without methionine, when the product is stored in cartridges under long-term conditions and accelerated conditions for up to 6 months.

The following compositions are tested:

### Composition A (2 Parallel Batches: AVE0010_09_894_A and AVE0010_09_897_A)

| | Specification | |
|---|---|---|
| | according to | Amount |
| Substance | pharmacopeia | per unit |
| AVE0010 | Sanofi-Aventis | 0.10 mg |
| Sodium acetate trihydrate | Ph. Eur./USP | 3.50 mg |
| m-Cresol | Ph. Eur./USP | 2.70 mg |
| 85% Glycerol | Ph. Eur./USP | 18.00 mg |
| 0.1N Hydrochloric acid | Ph. Eur./USP | ad pH 4.5 |
| 0.1N NaOH solution | Ph. Eur./USP | ad pH 4.5 |
| Water for injection (Wfl) | Ph. Eur./USP | ad 1.0 ml |

### Composition B (2 Parallel Batches: AVE0010_09_894_B and AVE0010_09_897_B)

| | Specification | |
|---|---|---|
| | according to | Amount |
| Substance | pharmacopeia | per unit |
| AVE0010 | Sanofi-Aventis | 0.10 mg |
| Sodium acetate trihydrate | Ph. Eur./USP | 3.50 mg |
| m-Cresol | Ph. Eur./USP | 2.70 mg |
| L-Methionine | Ph. Eur./USP | 3.00 mg |
| 85% Glycerol | Ph. Eur./USP | 18.00 mg |
| 0.1N Hydrochloric acid | Ph. Eur./USP | ad pH 4.5 |
| 0.1N NaOH solution | Ph. Eur./USP | ad pH 4.5 |
| Water for injection (Wfl) | Ph. Eur./USP | ad 1.0 ml |

The formulations are stored in units which are intended for clinical studies and for sales and distribution.

| Term | Description |
|---|---|
| Cartridge for | Cartridge, 3 ml colorless, type I glass |
| injection pen | (Ph. Eur.), SAP number 100922 |
| Crimped lid and | 7.5 mm |
| inserted within a | Crimped lid: aluminum |
| gray sealing disk | Sealing disk (exterior): isoprene rubber, |
| | material number 7773/35 |
| | Sealing disk (interior): bromobutyl rubber, |
| | material number 4780/40 |
| | Type I closure (Ph. Eur./USP) |
| | SAP number 164571 |
| Plunger | 9.2 × 11 mm |
| | Bromobutyl rubber, black |
| | SAP number 120521 |

Storage times, storage conditions, time points are summarized in the following table.

| | Test interval | | | |
|---|---|---|---|---|
| | (months) | | | |
| Condition | 0 | 1 | 3 | 6 |
| Long-term storage | | | | |
| +5 ± 3° C. | x | x | x | x |
| Accelerated conditions (temperature, humidity) | | | | |
| +25 ± 2° C./60 ± 5% RH | | x | x | x |
| +40 ± 2° C./75 ± 5% RH | | x | x | x |

The formulations are stored horizontally. RH means relative humidity. Time point 0 is the start of storage. The measurements at time point 0 are used as a reference for all conditions tested. During the tests, the samples are stored at ±5±3° C.

The physical and chemical stability of the stored formulations is determined with the help of the following tests:
Description
Clarity of the solution and color thereof
pH.
Chemical stability (purity and impurities, determined by HPLC, more particularly the proportion of oxidation products and of total impurities).
Proteins of high molecular weight, determined by HPSEC
Visible particles
Biological activity of the formulations

### Results

The formulations were studied separately for the parallel batches (894 and 897) with regard to the following parameters:
Biological activity of AVE0010. At 5° C. and 25° C., activity after 6 months was at least 96% of initial activity. The activities of the compositions according to the present invention were greater than the activities of the comparative compositions. At 40° C., activity after 6 months in the absence of methionine was approximately 43%. In the presence of methionine, activity was approximately 51% and thus clearly greater than in the absence of methionine.
Oxidation products. Measurements were carried out on an HPLC instrument (model: alliance) from Water Systems, using the 100% peak area method. For separation, a gradient of 0.1% TFA and acetonitrile as the mobile phase and a C18 reversed-phase column (Jupiter) as the stationary phase were used. At 5° C., the proportion of oxidized methionine Met(ox) in AVE0010 in the absence of methionine was 0.3%. At 25° C., the proportion was in the range of 0.6-0.8%, at 40° C. 1.3%. When the formulation comprised methionine, the proportion of oxidized methionine was distinctly lower. It was never more than 0.2% under all conditions tested. At 25° C., the proportion was thus approximately only ¼ to ⅓ of the content in the absence of methionine, even at 40° C. approximately only ⅙ (see FIGS. 1 and 2).
Proteins of high molecular weight. At 5° C., the proportion was between 0.1 and 0.3% and remained substantially unchanged during the entire storage time. At 25° C., the proportion rose in the absence of methionine to 0.9 and 1.3%, respectively. In the presence of methionine, the proportion was 0.4 to 0.5% and thus less than half as high. At 40° C., the proportion was in the absence of methionine 5.4% and 6.2%, respectively, while it was in the presence of methionine only 1.6 and 1.7%, respectively, and thus clearly lower (see FIGS. 3 and 4).
Total impurities. At 5° C., total impurities rose over the storage time of 6 months slightly from 1.2 to 1.8 or 1.9% (absence of methionine). When methionine was present, the rise was a little lower. At 25° C., a rise to 10.6% and 11.8%, respectively, was observed. In the presence of methionine, the values were below 10%. At 40° C., the proportion rose up to 54% (without methionine). When methionine was present, the proportion was approximately only 47% (see FIGS. 5 and 6).

The percentage values are the content values (percentage values of impurities) of the oxidation products, of total impurities, and of high-molecular-weight proteins (HMWP).

All values were determined by HPLC with the so-called 100% method. Here, in particular, it involves reversed-phase HPLC (C 18 column), in which a gradient method was used for the mobile phase:
a) 0.1% TFA, 15% ACN and b) 0.1% TFA, 75% ACN.

### Detection at 215 nm (UV).

The high-molecular-weight proteins (HMWP) were detected by HPSEC, described in European Pharmacopeia 6.0 for injectable insulin preparations.

The data are summarized in the following tables:

Mean values: AVE0010_09_894_A + B

| | AVE0010_09_894_A | | | | AVE0010_09_894_B | | | |
|---|---|---|---|---|---|---|---|---|
| | t₀ | 1 Mon. | 3 Mon. | 6 Mon. | t₀ | 1 Mon. | 3 Mon. | 6 Mon. |
| 5° C. | | | | | | | | |
| Total impurities | 1.2 | 1.5 | 2.1 | 1.8 | 1.1 | 1.3 | 1.5 | 1.7 |
| AVE0010 Test | 101.5 | 99.6 | 98.0 | 97.8 | 101.1 | 100.5 | 99.4 | 98.6 |
| Proteins of high molecular weight | 0.3 | 0.3 | 0.4 | 0.3 | 0.2 | 0.2 | 0.2 | 0.3 |
| Oxidation products | 0.3 | 0.4 | 0.4 | 0.3 | 0.1 | 0.2 | 0.1 | 0.1 |
| 25° C. | | | | | | | | |
| Total impurities | 1.2 | 3.0 | 6.4 | 11.8 | 1.1 | 2.5 | 5.7 | 9.8 |
| AVE0010 Test | 101.5 | 97.9 | 94.0 | 88.6 | 101.1 | 98.7 | 94.8 | 90.9 |
| Proteins of high molecular weight | 0.3 | 0.4 | 0.6 | 1.3 | 0.2 | 0.3 | 0.3 | 0.5 |
| Oxidation products | 0.3 | 0.4 | 0.5 | 0.8 | 0.1 | 0.2 | 0.2 | 0.2 |
| 40° C. | | | | | | | | |
| Total impurities | 1.2 | 13.4 | 34.3 | 54.1 | 1.1 | 12.1 | 30.4 | 46.8 |
| AVE0010 Test | 101.5 | 87.1 | 66.6 | 42.5 | 101.1 | 88.8 | 70.8 | 50.9 |
| Proteins of high molecular weight | 0.3 | 1.0 | 2.6 | 6.2 | 0.2 | 0.5 | 0.9 | 1.7 |
| Oxidation products | 0.3 | 0.6 | 0.9 | 1.3 | 0.1 | 0.2 | 0.2 | 0.2 |

Mean values: AVE0010_09_897_A + B

| | AVE0010_09_897_A | | | | AVE0010_09_897_B | | | |
|---|---|---|---|---|---|---|---|---|
| | t₀ | 1 Mon. | 3 Mon. | 6 Mon. | t₀ | 1 Mon. | 3 Mon. | 6 Mon. |
| 5° C. | | | | | | | | |
| Total impurities | 1.2 | 1.6 | 1.8 | 1.9 | 1.0 | 1.3 | 1.5 | 1.7 |
| AVE0010 Test | 99.2 | 98.2 | 97.5 | 96.7 | 99.5 | 99.2 | 98.0 | 97.1 |
| Proteins of high molecular weight | 0.1 | 0.1 | 0.1 | 0.2 | 0.1 | 0.1 | 0.1 | 0.1 |
| Oxidation products | 0.3 | 0.3 | 0.3 | 0.3 | 0.1 | 0.1 | 0.1 | 0.1 |
| 25° C. | | | | | | | | |
| Total impurities | 1.2 | 3.3 | 6.7 | 10.6 | 1.0 | 2.7 | 5.8 | 9.1 |
| AVE0010 Test | 99.2 | 96.6 | 92.8 | 87.4 | 99.5 | 97.8 | 93.6 | 90.0 |
| Proteins of high molecular weight | 0.1 | 0.2 | 0.5 | 0.9 | 0.1 | 0.1 | 0.2 | 0.4 |
| Oxidation products | 0.3 | 0.4 | 0.5 | 0.6 | 0.1 | 0.1 | 0.2 | 0.2 |
| 40° C. | | | | | | | | |
| Total impurities | 1.2 | 13.1 | 33.5 | 53.9 | 1.0 | 11.8 | 29.8 | 47.0 |
| AVE0010 Test | 99.2 | 86.8 | 66.5 | 42.6 | 99.5 | 88.0 | 70.7 | 51.0 |
| Proteins of high molecular weight | 0.1 | 0.8 | 2.2 | 5.4 | 0.1 | 0.4 | 0.9 | 1.6 |
| Oxidation products | 0.3 | 0.5 | 0.8 | 1.3 | 0.1 | 0.1 | 0.2 | 0.2 |

### Conclusion

The proportion of oxidation products, of proteins of high molecular weight, and of total impurities are, individually or together, a measure of the chemical integrity of the compositions. From the results described above with the example compositions, it follows that the liquid compositions according to the present invention comprising
a GLP-1 agonist or/and a pharmacologically tolerable salt thereof (more particularly AVE0010 or/and a pharmacologically tolerable salt thereof),
optionally at least one pharmaceutically acceptable excipient,
and methionine
   have improved stability or/and chemical integrity. The proportion of oxidized methionine, of total impurities, and of proteins of high molecular weight is lower in the compositions according to the present invention than in the comparative compositions. The composition according to the present invention (batches 894_B and 897_B) and the comparative compositions (batches 894_A and 897_A) differ in the presence/absence of methionine. Therefore, improved stability or/and chemical integrity can be ascribed to the methionine constituent in the compositions according to the present invention.

### EXAMPLE 2

In a further experiment, it was studied how sodium EDTA and histidine have an effect in a composition according to the present invention.

### Composition B (As In Example 1)

| | Specification | |
|---|---|---|
| | according to | Amount |
| Substance | pharmacopeia | per unit |
| AVE0010 | Sanofi-Aventis | 0.10 mg |
| Sodium acetate trihydrate | Ph. Eur./USP | 3.50 mg |
| m-Cresol | Ph. Eur./USP | 2.70 mg |
| L-Methionine | Ph. Eur./USP | 3.00 mg |
| 85% Glycerol | Ph. Eur./USP | 18.00 mg |
| 0.1N Hydrochloric acid | Ph. Eur./USP | ad pH 4.5 |
| 0.1N NaOH solution | Ph. Eur./USP | ad pH 4.5 |
| Water for injection (Wfl) | Ph. Eur./USP | ad 1.0 ml |

### Composition C

| | Specification | |
|---|---|---|
| | according to | Amount |
| Substance | pharmacopeia | per unit |
| AVE0010 | Sanofi-Aventis | 0.10 mg |
| Sodium acetate trihydrate | Ph. Eur./USP | 3.50 mg |
| Sodium EDTA | Ph. Eur./USP | 1.00 mg |
| m-Cresol | Ph. Eur./USP | 2.70 mg |
| L-Methionine | Ph. Eur./USP | 3.00 mg |
| L-Histidine | Ph. Eur./USP | 3.10 mg |
| 85% Glycerol | Ph. Eur./USP | 18.00 mg |
| 0.1N Hydrochloric acid | Ph. Eur./USP | ad pH 4.5 |
| 0.1N NaOH solution | Ph. Eur./USP | ad pH 4.5 |
| Water for injection (Wfl) | Ph. Eur./USP | ad 1.0 ml |

In a standard experimental design, rabbits were treated with composition B or C or a saline solution subcutaneously (s.c.) or intramuscularly (i.m.). In each case, half the rabbits were sacrificed after 24 hours or 120 hours in order to determine the acute or subacute effects of the administration histologically. Also, it was determined whether repair/regeneration of any changes occurred.

Following subcutaneous injection of composition C, the animals showed after 24 hours, in contrast to the saline control, a light to moderate inflammatory reaction in the subcutaneous connective tissue. After subcutaneous injection 120 hours earlier, a clear trend was observable for the observed changes to repair by a fibroblastic reaction. Thus, compatibility could still be rated as moderate (instead of as incompatible).

With composition B, the animals showed after subcutaneous injection no or minimal differences to the saline control (good compatibility).

After intramuscular injection of composition C, the animals exhibited muscular necrosis (multifocal or disseminated), clearly differing from the saline controls, in which only the site of injection was visible as a clearly circumscribed necrotic area. With composition C, mineralization of the necrotic muscular tissue was observed after 120 hours, visible even in a necropsy of the animals. Although small or focal mineralization at various sites in rabbits is not unusual, the mineralization after injection of composition C was clearly associated with the necrotic areas. Thus, the reversibility of the lesions caused by the injection is more than questionable. Based on these findings, composition C after intramuscular injection in rabbits was rated as incompatible.

Composition B after intramuscular injection showed good compatibility (no or minimal differences to the saline control).

From these data, it follows that composition B, compared to composition C, had an improved compatibility in intramuscular or subcutaneous administration. Subcutaneous injection is the preferred route of administration for the compositions comprising a GLP-1 agonist, more particularly AVE0010, described in this application.

Thus, the compositions according to the present invention, which comprise a GLP-1 agonist, more particularly AVE0010, can be free of EDTA or/and histidine. Likewise, the compositions according to the present invention can be substantially free of EDTA and histidine.

1. A liquid composition comprising a GLP-1 agonist and/or a pharmacologically tolerable salt thereof and at least one pharmaceutically acceptable excipient, wherein the composition comprises methionine.

2. The liquid composition as claimed in claim 1, wherein the composition comprises a pharmaceutically acceptable preservative.

3. The liquid composition as claimed in claim 1, wherein the composition comprises glycerol.

4. The liquid composition as claimed in claim 1, wherein the composition has a pH in the range from 3.5 to 5.

5. The liquid composition as claimed in claim 1, wherein the composition comprises methionine in an amount ranging from 0.5 mg/mL to 20 mg/mL.

6. The composition as claimed in claim 1, wherein the composition comprises methionine in an amount ranging from 1 mg/mL to 5 mg/mL.

7. The liquid composition as claimed in claim 1, wherein the composition exhibits chemical integrity after storage for 6 months at a temperature of ±25° C.

8. The liquid composition as claimed in claim 1, wherein the composition exhibits physical integrity after storage for 6 months at a temperature of ±25° C.

9. The liquid composition as claimed in claim 1, wherein the GLP-1 agonist is selected from the group consisting of GLP-1 and analogs and derivates thereof; exendin-3 and analogs and derivates thereof; and exendin-4 and analogs and derivates thereof.

10. The liquid composition as claimed in claim 1, wherein the composition comprises the following constituents:
(a) desPro³⁶exendin-4(1-39)-Lys₆-NH₂,
(b) sodium acetate,
(c) m-cresol,
(d) L-methionine,
(c) 85% glycerol,
(f) approximately 0.1 N hydrochloric acid, if adjustment to a pH of approximately 4.5 is required,
(g) approximately 0.1 N NaOH solution, if adjustment to a pH of approximately 4.5 is required, and
(h) water.

11. A method of treating diabetes mellitus in a patient in need thereof comprising administering to said patient a therapeutically effective amount of the composition of claim 1.

12. The method as claimed in claim 11, wherein said diabetes mellitus is type II diabetes.

13. A method for formulating a composition as claimed in claim 1, comprising formulating a GLP-1 agonist and/or a pharmacologically tolerable salt thereof with methionine and at least one pharmaceutically acceptable excipient.

14. The method of claim 11, further comprising the co-administration of metformin, a sulfonylurea, a glitazone, a long-acting insulin/insulin derivate, or a pharmacologically tolerable salt thereof.

15. The method of claim 14, further comprising an add-on therapy in patients where sufficient blood sugar control could not be achieved with metformin, a sulfonylurea, a glitazone, and/or a long-acting insulin/insulin derivate.

16. The method of claim 15, wherein the patient has an HbA1c value in the range from 7% to 10%.

17. A method of treating type II diabetes and/or obesity in a patient in need thereof, comprising administering to said patient a therapeutically effective amount of the composition of claim 1.

18. A method of supplementing a diet in order to improve blood sugar control in a patient in need thereof, comprising administering to said patient a supplementive amount of the composition of claim 1.

19. The method of claim 11, wherein said composition is administered to said patient once a day.


SEQUENCE LISTING (4 sequences)

SEQ ID NO: 1 - protein, 44 aa, synthetic construct
  REGION 1..44: note = Synthetic peptide
HGEGTFTSDLSKQMEEEAVRLFIEWLKNGGPSSGAPPSKKKKKK

SEQ ID NO: 2 - protein, 39 aa, synthetic construct
  REGION 1..39: note = Synthetic peptide
HGEGTFTSDLSKQMEEEAVRLFIEWLKNGGPSSGAPPPS

SEQ ID NO: 3 - protein, 39 aa, synthetic construct
  REGION 1..39: note = Synthetic peptide
HSDGTFTSDLSKQMEEEAVRLFIEWLKNGGPSSGAPPPS

SEQ ID NO: 4 - protein, 30 aa, synthetic construct
  REGION 1..30: note = Synthetic peptide
HAEGTFTSDVSSYLEGQAAKEFIAWLVKGR